(19) United States
(12) Patent Application Publication
(10) Pub. No.: US 2004/0009137 A1
(43) Pub. Date: Jan. 15, 2004

(54) Anhydrous, single phase, transfer resistant cosmetic lip compositions

(75) Inventor: Peter Jonathan Wyatt, Forest Hill, MD (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: THE PROCTER & GAMBLE COMPANY

(21) Appl. No.: 10/411,483

(22) Filed: Apr. 8, 2003

Related U.S. Application Data
(60) Provisional application No. 60/370,736, filed on Apr. 8, 2002.
(60) Provisional application No. 60/411,459, filed on Sep. 17, 2002.

(51) Int. Cl. A61K 7/06; A61K 7/11

(52) U.S. Cl. 424/70.12

(57) ABSTRACT

The present invention relates to cosmetic compositions that comprise:

(A) a mixture of:

(1) an organosiloxane resin; and

(2) a fluid diorganopolysiloxane polymer having a viscosity of from about 100,000 cSt to about 1,000,000 cSt at 25° C. wherein the ratio of (1) to (2) is from about 1:1 to about 20:1; and

(B) a volatile carrier

wherein said composition is present as a single phase and is an anhydrous liquid lip product. It has surprisingly been found that these compositions provide an improved feeling, long wear lip cosmetic product that resists transfer.

### CROSS REFERENCE TO RELATED APPLICATION

This application claims the benefit of U.S. Provisional Application No. 60/370,736, filed Apr. 8, 2002 and U.S. Provisional Application No. 60/411,459 filed Sep. 17, 2002.

### TECHNICAL FIELD

The present invention relates to anhydrous, single-phase cosmetic liquid lip compositions suitable for application to lips that comprise in a single phase an organosiloxane resin, a definitive fluid diorganopolysiloxane polymer, and a volatile carrier. Upon application the composition forms a thin, but, durable film resistant to transfer upon contact with objects such as clothing, towels, handkerchiefs and tissues.

### BACKGROUND

Transfer resistant lip products have gained increasing popularity over the last decade as consumers find themselves consumed with the stress of daily activities. These types of products allow consumers to apply the lip product fewer times in a day but still achieve a freshly applied appearance. One shortcoming of a number of these products, however, has been the undesirable feel on the lips that accompanies the use of such products. In particular, consumers complain of a tight feeling on the lips that typically results from the inclusion of film forming agents into these products that provides the long wear characteristic. Thus, there remains a need for a lip product that not only provides long wear for the consumer but at the same time provides a more pleasurable feel when applied and worn on the lips, e.g., a less tighter feeling product that is also less tacky.

### SUMMARY OF THE INVENTION

The cosmetic compositions of the present invention provide a durable film after application that resists degradation over time. Cosmetic compositions of the present invention comprise:

(A) a mixture of:

(1) an organosiloxane resin; and

(2) a fluid diorganopolysiloxane polymer having a viscosity of from about 100,000 cSt to about 1,000,000 cSt at 25° C. wherein the ratio of (1) to (2) is from about 1:1 to about 20:1; and

(B) a volatile carrier

wherein said composition is present as a single phase and is an anhydrous liquid lip product.

### DETAILED DESCRIPTION

The compositions of the present invention are anhydrous, single phase, transfer-resistant liquid lip cosmetic compositions. Applicants have found that compositions as detailed herein are particularly resistant against insult when subjected to the typical rigors of daily lip exposure.

As used herein, “comprising” means that other steps and ingredients can be added. This term encompasses the terms “consisting of” and “consisting essentially of”. The phrase “consisting essentially of” means that the composition may include additional ingredients, but only if the additional ingredients do not materially alter the basic and novel characteristics of the claimed compositions or methods.

The compositions of the present invention necessarily comprise the following ingredients.

Organosiloxane Resin

The compositions of the present invention comprise an organosiloxane resin. The resin may comprise combinations of R₃SiO_{1/2} “M” units, R₂SiO “D” units, RSiO_{3/2} “T” units, SiO₂ “Q” units in ratios to each other that satisfy the relationship RₙSiO_{(4-n)/2} where n is a value between 1.0 and 1.50 and R is a methyl group. Up to 5% of silanol or alkoxy functionality may also be present in the resin structure as a result of processing. The organosiloxane resins must be solid at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{1/2} “M” units and the quadrafunctional or SiO₂ “Q” units, otherwise known as “MQ” resins as disclosed in U.S. Pat. No. 5,330,747, Krzysik, issued Jul. 19, 1994, incorporated herein by reference. In the present invention the ratio of the “M” to “Q” functional units is preferably about 0.7 and the value of n is 1.2. Organosiloxane resins such as these are commercially available such as Wacker 803 and 804 available from Wacker Silicones Corporation of Adrian Mich., and G. E. SR1000 from the General Electric Company.

The organosiloxane resins are used in the present invention at levels from about 10% to about 95%, preferably from about 55% to about 80% and most preferably from about 60% to about 70% of the total amount of organosiloxane resin and fluid diorganopolysiloxane polymers.

Fluid Diorganopolysiloxane Polymer

The present invention employs a fluid diorganopolysiloxane polymer that is combined with the organosiloxane resin disclosed above. Applicants have found that suitable polymers exhibit a viscosity of from about 100,000 to about 1,000,000 cSt at 25° C. Preferred fluid diorganopolysiloxane polymers have viscosities of from about 200,000 to about 1,000,000 cSt at 25° C. and even more preferred of from about 300,000 to about 1,000,000 cSt at 25° C., and most preferred of from about 300,000 to about 950,000 cSt at 25° C.

The fluid diorganopolysiloxane polymers of the present invention comprise repeating units, wherein said units correspond to the formula (R₂SiO), where R is a monovalent hydrocarbon radical containing from 1 to 6 carbon atoms, preferably selected from the group consisting of methyl, ethyl, propyl, isopropyl, butyl, isobutyl, t-butyl, amyl, hexyl, vinyl, allyl, cyclohexyl, phenyl, fluoroalkyl and mixtures thereof. The diorganopoylsiloxane fluids employed in the present invention may contain one or more of these hydrocarbon radicals as substituents on the siloxane polymer backbone. The diorganopolysiloxane fluids may be terminated by triorganosilyl groups of the formula (R₃Si) where R is a radical selected from the group consisting of monovalent hydrocarbons containing from 1-6 carbon atoms, hydroxyl groups, alkoxyl groups and mixtures thereof. When a diorganopolysiloxane polymer fluid is present, it is essential that it be compatible in solution with the organosiloxane resin material and the volatile carrier. The term “compatible” refers to the formation of a single phase solution when the diorganopolysiloxane fluid, organosiloxane resin and volatile carrier are mixed together in ratios required for a specific formulation. Therefore, the compositions are considered to be “single phase” in nature as well. A particularly preferred fluid diorganopolysiloxane polymer is poly(dimethylsiloxane), herein referred to as PDMS. A preferred fluid diorganopolysiloxane polymer is DM300,000, which is a 300,000 cSt dimethicone commercially available from Wacker Silicones. This DM300,000 and other suitable fluid diorganopolysiloxane polymers have the following structural formula wherein n=2000 to 2500.

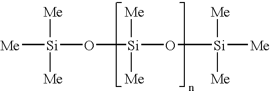

Volatile Carrier

In the present invention, the combination of the organosiloxane resin and fluid diorganosiloxane polymer above must be easily transferred to the lip surface using a package/applicator. To achieve delivery, it is necessary that this combination above be incorporated into a carrier, specifically a volatile carrier which quickly volatilizes from the surface of the lips leaving the above-discussed thin-durable film. The volatile carrier must solubilize the organosiloxane resin and the fluid diorganosiloxane polymer.

The volatile carrier comprises from about 10% to about 90%, preferably from about 15% to about 80%, and most preferably from about 20% to about 70% of the composition. The volatile carrier of the present invention are selected from the group consisting of volatile hydrocarbons, volatile silicones and mixtures thereof.

Hydrocarbon oils useful in the present invention include those having boiling points in the range of 60-260° C., more preferably hydrocarbon oils having from about C₅ to about C₂₀ chain lengths, most preferably C₇ to C₁₆ isoparaffins. Of these isoparriffins most preferred are selected from the group consisting of isododecane, isohexadecane, isoeocosane, 2,2,4-trimethylpentane, 2,3-dimethylhexane and mixtures thereof. Most preferred is isododecane, which is also known as 2,2,4,6,6-pentamethylheptane. Isododecane that is suitable for inclusion in the compositions of the present invention is available from a number of sources, e.g., from Presperse, Inc. as Permethyl 99A.

Preferred volatile silicone fluids include cyclomethicones having 4, 5, and 6 member ring structures corresponding to the formula:

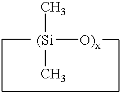

where X is from about 3 to about 6. Said volatile silicones include 244 Fluid, 344 Fluid and 245 Fluid, and 345 Fluid all from Dow Corning Corporation.

Thickeners/Structure Builders

The cosmetic composition of the present invention may also comprise thickeners and/or structure builders which have the function of dispersing pigments in addition to building viscosity. Thickeners and/or structure builders useful for the present invention include, but are not limited to, organically modified clays. Organically modified clays useful for the present invention include, but are not limited to, hectorite and/or bentonite.

When organically modified clays are present, the compositions comprise less than about 3.5%, by weight of the composition, more preferably, from about 1.5% to about 3%, and most preferably from about 2% to about 3%.

Pigments

Pigments suitable for use herein are all inorganic and organic colors/pigments suitable for use in lip composition compositions. These are usually aluminum, barium or calcium salts or lakes. Lakes are either a pigment that is extended or reduced with a solid diluent or an organic pigment that is prepared by the precipitation of a water-soluble dye on an adsorptive surface, which usually is aluminum hydrate. A lake also forms from precipitation of an insoluble salt from an acid or basic dye. Calcium and barium lakes are also used herein.

Preferred lakes of the present invention are Red 3 Aluminum Lake, Red 21 Aluminum Lake, Red 27 Aluminum Lake, Red 28 Aluminum Lake, Red 33 Aluminum Lake, Yellow 5 Aluminum Lake, Yellow 6 Aluminum Lake, Yellow 10 Aluminum Lake, Orange 5 Aluminum Lake and Blue 1 Aluminum Lake, Red 6 Barium Lake, Red 7 Calcium Lake, Red 30 Talc Lake, and Red 30 Aluminum Lake.

Other colors and pigments can also be included in the lip compositions, such as dyes and pearls, titanium oxides, Red 6, Red 21, Blue 1, Orange 5, and Green 5 dyes, chalk, talc, iron oxides and titanated micas.

Compositions of the present invention contain sufficient pigments to provide the look sought by the user. The pigments are used herein at levels relative to the level of the fluid diorganopolysiloxane polymers disclosed above. This level is expressed as a ratio of the combination of fluid diorganopolysiloxane polymer and organosiloxane resin to pigment. In the present invention this ratio is from about 1:1 to about 30:1, preferably from about 1.5:1 to about 15:1, and most preferably from about 2:1 to about 10:1.

Additionally, the cosmetic compositions of the present invention are anhydrous. As used herein “anhydrous” means that the compositions comprise less than about 5%, by weight of the composition, of water, more preferably less than about 2%, and even more preferably less than about 1%.

Waxes

Waxes may be used in the present invention provided they are used at levels which does not interfere with film formation process.

Waxes are defined as lower-melting organic mixtures or compounds of high molecular weight, solid at room temperature and generally similar in composition to fats and oils except that they contain no glycerides. Some are hydrocarbons, others are esters of fatty acids and alcohols. Waxes useful in the present invention are selected from the group consisting of animal waxes, vegetable waxes, mineral waxes, various fractions of natural waxes, synthetic waxes, petroleum waxes, ethylenic polymers, hydrocarbon types such as Fischer-Tropsch waxes, silicone waxes, and mixtures thereof.

The specific waxes useful in the present invention are selected from the group consisting of synthetic waxes, ozokerite, jojoba esters, “Unilins”, available from Petrolite Corporation, “Ganex” alkylated polyvinylpyrrolidines available from the ISP Company, fatty alcohols from C22 to C50 and mixtures thereof. Synthetic waxes include those disclosed in Warth, Chemistry and Technology of Waxes, Part 2, 1956, Reinhold Publishing; herein incorporated by reference. The waxes most useful herein are selected from the C₈ to C₅₀ hydrocarbon waxes. Such waxes include long chained polymers of ethylene oxide combined with a dihydric alcohol, namely polyoxyethylene glycol. Such waxes include carbowax available from Carbide and Carbon Chemicals company. Other synthetic waxes include long-chained polymers of ethylene with OH or other stop length grouping at end of chain. Such waxes include the Fischer-Tropsch waxes as disclosed in the text disclosed above at pages 465-469 and include Rosswax, available from Ross company and PT-0602 available from Astor Wax Company.

The cosmetic compositions of the present invention are also in the form of liquid lip products. As such, the compositions comprise less than about 2%, by weight of the composition, of waxes, more preferably, less than about 1%, and most preferably less than about 0.5%.

Other Ingredients

There are a number of other ingredients approved for use in the cosmetic art that may be used in compositions of the present invention. Such ingredients are those approved for use in cosmetics and can be found listed in reference books such as the CTFA Cosmetic Ingredient Handbook, Second Edition, The Cosmetic, Toiletries, and Fragrance Association, Inc. 1988, 1992. Said materials may be used provided their inclusion does not significantly disrupt the composition once it has been applied wherein a film has been formed. Said ingredients include waxes, fragrances, flavor oils, skin care ingredients such as sunscreen, emulsifiers and the like. Hypoallergenic compositions can be made into the present invention where said compositions do not contain fragrances, flavor oils, lanolin, sunscreens, particularly PABA, or other sensitizers and irritants.

Complimentary products may be used in conjunction with the present invention to compliment the composition and improve its aesthetic appeal to the user. In particular, it is intended that the cosmetic compositions of the present invention may be paired in a lip cosmetic kit with such a complementary product.

It is specifically envisioned that the complementary products used in the present invention manner wherein such a product is applied over the film formed after application of the cosmetic composition of the present invention. For example in the case of lip compositions, a complimentary product may be utilized to enhance the gloss and shine of the lips and provide a lubricious feeling. Such products, otherwise known as an “overcoat” or “topcoat” may be in a stick or liquid form and can include any that are commercially available or to be developed, provided the aggregate of the materials comprising the overcoat does not significantly disrupt the composition of the present invention. The overcoat compositions may be clear or transparent or may contain dyes and/or colorants that when viewed along with the overcoat, produce a desired color.

One such material that has been shown to be quite useful in formulating complimentary products is polyol polyesters, such as sucrose polyesters (herein referred to as SPE'S). SPE's are synthesized molecules derived from sugar and vegetable oil and have been extensively disclosed in the patent literature in context of a non-digestible oils. Such compositions are generally disclosed in U.S. Pat. No. 3,600,186, issued Aug. 17, 1971; U.S. Pat. No. 4,005,195, issued Jan. 25, 1977; U.S. Pat. No. 4,005,196, issued Jan. 25, 1977; all assigned to the Procter & Gamble Company and all herein incorporated by reference.

It has found that overcoat compositions having a significant level of SPE'S are incompatible with the lip composition of the present composition wherein upon their application, the cosmetic composition of the present invention is not disrupted.

In a preferred embodiment of the present invention, a complementary product comprising a topcoat composition comprises a safe and effective amount of a polymeric vegetable oil emollient. More preferably, the polymeric vegetable oil emollient is a copolymer of Brassica Campestris and Aleurites Fordii oil (also known as polytriglyceryl erucate/eliostearate), which is an unsaturated complex ester ranging in molecular weight from 25,000 to 110,000 (or 6500 to about 7500 daltons). This emollient is commercially available from Tri-K under the tradename Glossamer L-6600 and allows the topcoat to form a protective film over the cosmetic compositions of the present invention. Such a topcoat composition aids in providing a creamier feel to the applied basecoat and topcoat and also enhances the water and rub-off resistance of the cosmetic composition as well as the topcoat composition. In more preferred embodiments, the polymeric vegetable oil emollient is present in an amount of from about 0.1% to about 50%, more preferably, from about 2% to about 20%, and most preferably, from about 4% to about 15%, by weight of the topcoat composition.

### Method of Using the Invention

The method of the present invention is straightforward. The user applies the composition of the present invention from a suitable liquid cosmetic applicator directly onto the skin. One such applicator used for liquid products is a liquid pen package disclosed in British Patent 21198037, issued May 9, 1990, assigned to Mitsubishi Pencil Co., Ltd. of Japan. An alternative package is one where an wand is dipped into a reservoir wherein the composition on the tip of the wand is applied to the skin surface. Such packages are disclosed in Japanese Utility Model 64 000822 Y2, to Shiseido.

Another cosmetic dispenser that is useful for the present invention is a unidirectional twist-up dispensing device with incremental dosing as disclosed in U.S. Pat. No. 5,851,079, issued on Dec. 22, 1998 to Richard L Horstman et al. Such a twist-up dispensing device can include a hollow housing defining a chamber having an open dispensing end and a piston located within the chamber being limited to translational movement within the chamber. The piston preferably having a threaded rod extending therefrom that engages with a threaded aperture in an actuator such that advancement of the piston toward the dispensing end occurs when the actuator is rotated. Rotation of the actuator causes the product to be dispensed from the dispensing end. An applicator is preferably attached to the dispensing end of the housing in fluid communication with the chamber wherein the product is dispensed through the applicator. The applicator can comprise a ferrule and an application portion wherein the ferrule is attached to the dispensing end of the housing and the application portion has at least one orifice located therein. Several versions of applicators can be utilized including, for example, a fiber brush or an application surface having flocking thereon. Flocking is a mat of thin, short, plastic fibers substantially perpendicular to the application surface. The bristles of a fiber brush are preferably tapered and made of a plastic material. Alternatively, the user may use a more traditional applicator or implement known in the art.

As stated above, the user applies the composition wherein the user allows the composition to dry before subjecting the composition to insult. Once the composition is dried, a complimentary product such as the topcoat product disclosed above may be applied over the dried product to provide the user with an asthetically pleasing affect. Topcoat compositions may utilize the same dispensing device as discribed for use of the compositions of the present invention.

The compositions of the present invention may be removed by applying petrolatum or a dimethicone-based cosmetic remover and by rubbing the area gently with a tissue to remove the cosmetic.

### EXAMPLES

The following examples illustrate the examples of the claimed cosmetic compositions of the present invention but are not intended to be limiting thereof:

### Example 1

| Ingredients | Weight (%) |
|---|---|
| Group A: | |
| Polydimethylsiloxane¹ | 10 |
| Bentone Gel² | 20 |
| Isododecane³ | 40 |
| Group B: | |
| Organosiloxane Resin⁴ | 20 |
| Red #7 Calcium Lake | 5 |
| Titanated Mica⁵ | 3 |
| Titanium Dioxide | 1.5 |
| Black Iron Oxide | .5 |

¹300,000 cSt polydimethylsiloxane available as DM300000 from Wacker Silicones. May also substitute polydimethylsiloxanes with viscosities at 25° C. of 400,000 cSt, 500,000 cSt, 600,000 cSt, 700,000 cSt, 800,000 cSt, and 900,000 cSt.
²Bentone Gel ISD available from Elementis
³Permethyl 99A available from Permethyl Corporation.
⁴MQ Resin (0.7:1 M:Q) available as SR1000 from General Electric
⁵Flamenco Superpearl available from Engelhard
Combine Group A ingredients together ina beaker and mix with a propeller mixer until the mixture is homogeneous. Add Group B ingredients to the Group A Mixture and dry mix the mixture to roughly incorporate the dry powders. Then homogenize the formulation until all pigments are full dispersed. Transfer the resulting fluid to individual packages.

Combine Group A ingredients together in a beaker and mix with a propeller mixer until the mixture is homogeneous. Add Group B ingredients to the Group A Mixture and dry mix the mixture to roughly incorporate the dry powders. Then homogenize the formulation until all pigments are full dispersed. Transfer the resulting fluid to individual packages.

### Example 2

| Ingredients | Weight (%) |
|---|---|
| Group A: | |
| Polydimethylsiloxane¹ | 8 |
| Bentone Gel² | 30 |
| Isododecane³ | 35 |
| Group B: | |
| Organosiloxane Resin⁴ | 17 |
| Red #7 Calcium Lake | 5 |
| Titanated Mica⁵ | 3 |
| Titanium Dioxide | 1.5 |
| Black Iron Oxide | .5 |

¹300,000 cSt polydimethylsiloxane available as DM300000 from Wacker Silicones. May also substitute polydimethylsiloxanes with viscosities at 25° C. of 400,000 cSt, 500,000 cSt, 600,000 cSt, 700,000 cSt, 800,000 cSt, and 900,000 cSt.
²Bentone Gel ISD available from Elementis
³Permethyl 99A available from Permethyl Corporation.
⁴MQ Resin (0.7:1 M:Q) available as SR1000 from General Electric
⁵Flamenco Superpearl available from Engelhard
Combine Group A ingredients together ina beaker and mix with a propeller mixer until the mixture is homogeneous. Add Group B ingredients to the Group A Mixture and dry mix the mixture to roughly incorporate the dry powders. Then homogenize the formulation until all pigments are full dispersed. Transfer the resulting fluid to individual packages.

### Example 3

| Ingredients | Weight (%) |
|---|---|
| Group A: | |
| Polydimethylsiloxane¹ | 10 |
| Bentone Gel² | 20 |
| Isododecane³ | 30 |
| Group B: | |
| Organosiloxane Resin⁴ | 20 |
| Red #7 Calcium Lake | 10 |
| Titanated Mica⁵ | 6 |
| Titanium Dioxide | 3 |
| Black Iron Oxide | 1 |

¹300,000 cSt polydimethylsiloxane available as DM300000 from Wacker Silicones. May also substitute polydimethylsiloxanes with viscosities at 25° C. of 400,000 cSt, 500,000 cSt, 600,000 cSt, 700,000 cSt, 800,000 cSt, and 900,000 cSt.
²Bentone Gel ISD available from Elementis
³Permethyl 99A available from Permethyl Corporation.
⁴MQ Resin (0.7:1 M:Q) available as SR1000 from General Electric
⁵Flamenco Superpearl available from Engelhard
Combine Group A ingredients together in a beaker and mix with a propeller mixer until the mixture is homogeneous. Add Group B ingredients to the Group A Mixture and dry mix the mixture to roughly incorporate the dry powders. Then homogenize the formulation until all pigments are full dispersed. Transfer the resulting fluid to individual packages.

### Example 4

| Ingredients | Weight (%) |
|---|---|
| Group A: | |
| Polydimethylsiloxane¹ | 10 |
| Bentone Gel² | 20 |
| Cyclomethicone³ | 40 |
| Group B: | |
| Organosiloxane Resin⁴ | 20 |
| Red #7 Calcium Lake | 5 |
| Titanated Mica⁵ | 3 |
| Titanium Dioxide | 1.5 |
| Black Iron Oxide | .5 |

¹500,000 cSt polydimethylsiloxane available as DM300000 from Wacker Silicones. May also substitute polydimethylsiloxanes with viscosities at 25° C. of 400,000 cSt, 500,000 cSt, 600,000 cSt, 700,000 cSt, 800,000 cSt, and 900,000 cSt.
²VS-5 PC available from Elementis
³DC245 available from Dow Corning
⁴MQ Resin (0.7:1 M:Q) available as SR1000 from General Electric
⁵Flamenco Superpearl available from Engelhard
Combine Group A ingredients together in a beaker and mix with a propeller mixer until the mixture is homogeneous. Add Group B ingredients to the Group A Mixture and dry mix the mixture to roughly incorporate the dry powders. Then homogenize the formulation until all pigments are full dispersed. Transfer the resulting fluid to individual packages.

### Examples 5-10

Complementary topcoat compositions that can be paired with the basecoat compositions of the present invention are made by combining the following ingredients.

| | Exam- | Exam- | Exam- | Exam- | Exam- | Exam- |
|---|---|---|---|---|---|---|
| Ingredient | ple 5 | ple 6 | ple 7 | ple 8 | ple 9 | ple 10 |
| Sucrose Poly- | 81.800 | 83.800 | 75.800 | 86.800 | 74.800 | 70.800 |
| cottonseedate | | | | | | |
| Propylparaben | 0.100 | 0.100 | 0.100 | 0.100 | 0.100 | 0.100 |
| Beeswax | 5.500 | 4.500 | 8.500 | 5.500 | 5.500 | 7.500 |
| Ozokerite | 5.500 | 4.500 | 8.500 | 5.500 | 5.500 | 7.500 |
| Propyl Gallate | 0.050 | 0.050 | 0.050 | 0.050 | 0.050 | 0.050 |
| Glossamer | 7.000 | 7.000 | 7.000 | 2.000 | 14.000 | 14.000 |
| L-6600 | | | | | | |
| Ethylene | 0.050 | 0.050 | 0.050 | 0.050 | 0.050 | 0.050 |
| Brassylate | | | | | | |
| Total | 100.000 | 100.000 | 100.000 | 100.000 | 100.000 | 100.000 |

Once ingredients are mixed, topcoat compositions are poured into individual packages.

### Examples 11-16

Complementary topcoat compositions that can be paired with the basecoat compositions of the present invention are made by combining the following ingredients.

| | Exam- | Exam- | Exam- | Exam- | Exam- | Exam- |
|---|---|---|---|---|---|---|
| Ingredient | ple 11 | ple 12 | ple 13 | ple 14 | ple 15 | ple 16 |
| Sucrose Poly- | 76.800 | 78.800 | 65.800 | 71.800 | 73.600 | 55.800 |
| cottonseedate | | | | | | |
| Propylparaben | 0.100 | 0.100 | 0.100 | 0.100 | 0.100 | 0.100 |
| Beeswax | 5.500 | 4.500 | 8.500 | 5.500 | 5.500 | 7.500 |
| Ozokerite | 5.500 | 4.500 | 8.500 | 5.500 | 5.500 | 7.500 |
| Propyl Gallate | 0.050 | 0.050 | 0.050 | 0.050 | 0.050 | 0.050 |
| Glossamer | 7.000 | 7.000 | 7.000 | 2.000 | 14.000 | 14.000 |
| L-6600 | | | | | | |
| Ethylene | 0.050 | 0.050 | 0.050 | 0.050 | 0.050 | 0.050 |
| Brassylate | | | | | | |
| Silk Mica | — | 3.500 | — | — | 0.100 | 0.500 |
| Gemtone | — | — | — | 1.500 | 0.100 | |
| Tan Opal | | | | | | |
| Colorona | 0.500 | — | — | — | 0.100 | 0.500 |
| Bronze | | | | | | |
| Black Mica | — | 0.500 | — | — | 0.100 | — |
| Cloisonne Nu- | — | — | — | — | 0.100 | 0.200 |
| Antique Bronze | | | | | | |
| Colorona | — | — | 9.000 | — | 0.100 | 1.000 |
| Magenta | | | | | | |
| Cloisonne | — | — | — | — | 0.100 | — |
| Nu-Antique | | | | | | |
| Rouge Flambe | | | | | | |
| Gemtone | 4.500 | — | — | — | 0.100 | 4.500 |
| Garnet | | | | | | |
| Timica Extra- | — | 0.500 | — | — | 0.100 | |
| Large Sparkle | | | | | | |
| Flamenco Gold | — | — | — | 13.500 | 0.100 | 8.000 |
| Timiron MP 24 | — | — | 1.000 | — | 0.100 | — |
| Cloisonne | — | 0.500 | — | — | 0.100 | 0.300 |
| Rouge Flambe | | | | | | |
| Total | 100.000 | 100.000 | 100.000 | 100.000 | 100.000 | 100.000 |

Once ingredients are mixed, topcoat compositions are poured into individual packages.

What is claimed is:

1. A cosmetic composition comprising:
(A) a mixture of:
(1) an organosiloxane resin; and
(2) a fluid diorganopolysiloxane polymer having a viscosity of from about 100,000 cSt to about 1,000,000 cSt at 25° C. wherein the ratio of (1) to (2) is from about 1:1 to about 20:1; and
(B) a volatile carrier
wherein said composition is present as a single phase and is an anhydrous liquid lip product.

2. The cosmetic composition of claim 1 further comprising an organically modified clay.

3. The cosmetic composition of claim 2 wherein the organically modified clay is hectorite, bentonite, or mixtures thereof.

4. The cosmetic composition of claim 1 wherein the organosiloxane resin comprises R₃SiO_{1/2} “M” units, R₂SiO “D” units, RSiO_{3/2} “T” units, SiO₂ “Q” units and mixtures thereof in a ratio selected to satisfy the relationship RₙSiO_{(4-n)/2}, wherein n is from about 1.0 to about 1.50 and R is a methyl group, wherein the resin is solid at about 25° C., has a molecular weight range from about 1,000 to about 10,000 grams/mole and is soluble in organic solvents.

5. The cosmetic composition of claim 1 wherein the organosiloxane resin comprises R₃SiO_{1/2} “M” units and SiO₂ “Q” units wherein the ratio of R₃SiO_{1/2} to SiO₂ is about 0.7 wherein n is about 1.2.

6. The cosmetic composition of claim 1 wherein the fluid diorganopolysiloxane polymer comprises repeating units of the formula (R₂SiO), where R is a hydrocarbon side group containing from 1 to 6 carbon atoms.

7. The cosmetic composition of claim 6 wherein said hydrocarbon side group is selected from the group consisting of methyl, ethyl, propyl, isopropyl, butyl, isobutyl, t-butyl, amyl, hexyl, vinyl, allyl, cyclohexyl, phenyl, fluoroalkyl and mixtures thereof.

8. The cosmetic composition of claim 7 wherein the fluid diorganopolysiloxane polymer is terminated by triorganosilyl groups of the formula (R₃Si) where R is a monovalent hydrocarbon radical selected from the group consisting of 1 to 6 carbon atoms, hydroxyl groups, an alkoxyl groups and mixtures thereof.

9. The cosmetic composition of claim 8 wherein the fluid diorganopolysiloxane polymer is a polydimethyl siloxane.

10. The cosmetic composition of claim 1 wherein the diorganopolysiloxane polymer has a viscosity of from about 200,000 to about 1,000,000 cSt at 25° C.

11. The cosmetic composition of claim 1 wherein the volatile carrier is selected from the group consisting of hydrocarbon oils, silicone oils and mixtures thereof.

12. The cosmetic composition of claim 1 wherein the volatile carrier is isododecane.

13. A method of applying the composition of claim 1 to lips, said method comprising the steps of:
a. applying the composition from a suitable cosmetic applicator directly onto the lips; and
b. allowing said composition to form a dry film on the lips before subjecting the film to insult.

14. The method of claim 15 that comprises the additional step of applying a complimentary product comprising a topcoat composition on said dried film where upon applying said complimentary product said film is not disrupted.

15. The method of claim 16 wherein said topcoat composition comprises a safe and effective amount of a polymeric vegetable oil emollient.

16. A lip cosmetic kit comprising:
a. a cosmetic composition comprising:
1. a mixture of:
(A) an organosiloxane resin; and
(B) a fluid diorganopolysiloxane polymer having a viscosity of from about 100,000 cSt to about 1,000,000 cSt at 25° C. wherein the ratio of (1) to (2) is from about 1:1 to about 20:1; and
2. a volatile carrier; wherein said cosmetic composition is present as a single phase and is an anhydrous liquid lip product; and
b. a complimentary product comprising a topcoat composition that comprises a safe and effective amount of a polymeric vegetable oil emollient.
